(19) United States
(12) Patent Application Publication
(10) Pub. No.: US 2026/0294960 A1
(43) Pub. Date: Oct. 1, 2026

(54) METHODS FOR GENERATING ALGINATE HYDROGELS AND HYDROGEL SYSTEMS

(71) Applicant: The Regents of the University of Michigan, Ann Arbor, MI (US)

(72) Inventors: Tianxiang Liu, Ann Arbor, MI (US); Jihpeng Sun, Ann Arbor, MI (US); Rong Ma, Ann Arbor, MI (US); Sungwan Park, Ann Arbor, MI (US)

(21) Appl. No.: 19/636,580

(22) Filed: Apr. 1, 2026

Related U.S. Application Data
(60) Provisional application No. 63/781,725, filed on Apr. 1, 2025.

(51) Int. Cl. A61K 31/738; C08J 3/075; C08J 3/24

(52) U.S. Cl. CPC A61K 31/738; C08J 3/075; C08J 3/24; C08J 2205/022; C08J 2207/10

(57) ABSTRACT

The present disclosure provides methods for modifying an alginate hydrogel and methods for generating a modified alginate hydrogel (e.g., a modified alginate microgel), and systems, compositions, and uses thereof. Particularly disclosed herein are methods which comprise incubating the alginate hydrogel in a solution comprising a second crosslinker to form a modified alginate hydrogel, wherein the second crosslinker comprises a different cation or cation mixture from a first crosslinker used to form the alginate hydrogel.

### CROSS-REFERENCE TO RELATED APPLICATIONS

This application claims the benefit of U.S. Provisional Application No. 63/781,725, filed Apr. 1, 2025, the content of which is herein incorporated by reference in its entirety.

### STATEMENT REGARDING FEDERALLY SPONSORED RESEARCH OR DEVELOPMENT

This invention was made with government support under 2243104 awarded by the National Science Foundation. The government has certain rights in the invention.

### FIELD

The present disclosure provides methods for modifying an alginate hydrogel and methods for generating a modified alginate hydrogel (e.g., a modified alginate microgel).

### BACKGROUND

Hydrogels, composed of natural or synthetic crosslinked polymers with high water content, have garnered significant attention due to their versatile properties and broad applications. Their structure, maintained by both physical and covalent crosslinking, forms a three-dimensional hydrophilic porous network that enables exceptional water uptake and retention. Recent advancements have enabled the preparation of hydrogel particles at the micrometer scale, offering distinct advantages over their bulk counterparts in drug delivery and other therapeutic applications. Their small, injectable form factor allows for minimally invasive administration via methods such as percutaneous injections using syringes or catheters. Consequently, these hydrogel microparticles, or microgels, are particularly appealing for controlled drug release because of their injectability, low invasiveness, and high surface area to volume ratio.

Achieving programmable release of payloads with diverse molecular weights at distinct rates typically require complex polymer designs that can compromise the accessibility and biocompatibility of the delivery system. For example, varying the ratios of stable and cleavable crosslinkers enables compositional control over payload release, as cleavable crosslinkers degrade more rapidly to increase the encapsulant release rate. Other strategies include modifying polymer chains with functional groups to adjust the hydrogel's affinity for encapsulated drugs, further tuning release characteristics. Moreover, incorporating multiple polymer constituents can introduce additional complexity into the release profile. The use of specialty covalent crosslinkers or custom polymer mixtures, however, adds complexity, limits accessibility, and may compromise the biocompatibility of the delivery system. Therefore, it is desirable to develop techniques that fine-tune payload release profiles without the need for additional chemical agents.

### SUMMARY

In one aspect, disclosed herein are methods of modifying and generating alginate hydrogel. In some embodiments, the alginate hydrogel exhibits modified swelling rates, swelling ratios, stability, crosslinking strength and/or density, cargo release profiles, or combination thereof.

In some embodiments, the methods comprise incubating an alginate hydrogel in a solution comprising a second crosslinker to form a modified alginate hydrogel, wherein the second crosslinker has a different cation or cation mixture from a first crosslinker used to form the alginate hydrogel. In some embodiments, the modified alginate hydrogel exhibits modified swelling rates, swelling ratios, stability, crosslinking strength and/or density, cargo release profiles, or combination thereof, as compared to an alginate hydrogel not incubated with the second crosslinker.

In some embodiments, the methods further comprise rinsing the modified alginate hydrogel, swelling the modified alginate hydrogel, or a combination thereof.

In some embodiments, the alginate hydrogel is an alginate microgel.

In some embodiments, the first crosslinker comprises calcium. In some embodiments, the first crosslinker further comprises at least one additional cation. In some embodiments, the second crosslinker comprises magnesium or sodium. In some embodiments, the second crosslinker lacks calcium.

In some embodiments, the methods further comprise generating the alginate hydrogel with the first crosslinker.

In some embodiments, generating the alginate hydrogel comprises adding alginate to a solution comprising the first crosslinker. In some embodiments, the first crosslinker is calcium. In some embodiments, the calcium is provided in a solution at 1 to 10 wt % calcium. In some embodiments, the alginate hydrogel is generated using an emulsion-based method, a microfluidic method, lithography, or a centrifugal-force-driven method. In select embodiments, the alginate hydrogel is generated using a centrifugal-force-driven method.

In some embodiments, the alginate hydrogel further comprises an active agent. In some embodiments, generating the alginate hydrogel further comprises adding an active agent to the first crosslinker or alginate prior to crosslinking. In some embodiments, the solution comprising the second crosslinker further comprises an active agent. In some embodiments, the methods further comprise incubating the modified alginate hydrogel with an active agent.

In some embodiments, the methods further comprise coating at least a portion of the modified alginate hydrogel or particles thereof. In some embodiments, the solution comprising the second crosslinker further comprises a coating agent. In some embodiments, the coating agent is polyethylene glycol (PEG).

In one aspect, disclosed herein are modified alginate hydrogels formed by the methods disclosed herein. In some embodiments, the modified alginate hydrogel is an alginate microgel. In some embodiments, the modified alginate hydrogel or particles thereof are at least partially coated with polyethylene glycol (PEG). In some embodiments, the modified alginate hydrogel comprises an active agent.

In one aspect, disclosed herein are hydrogel systems comprising at least one modified alginate hydrogel or alginate hydrogel as described herein. In some embodiments, they hydrogel system comprises two or more modified alginate hydrogels or alginate hydrogels as disclosed herein. In some embodiments, at least one of the two or more modified alginate hydrogels were prepared with different second crosslinkers. In some embodiments, any one or more or all of at least one modified alginate hydrogels, or particles thereof, are at least partially coated, e.g., with polyethylene glycol (PEG). In some embodiments, any one or more or all of the least one modified alginate hydrogel or alginate hydrogel comprises an active agent. In some embodiments, each of the at least one modified alginate hydrogel or alginate hydrogel comprises a same or different active agent.

In one aspect, disclosed herein are methods of delivering an active agent to a subject in need thereof. In some embodiments, the methods comprise administering to the subject a modified alginate hydrogel or a hydrogel system as disclosed herein.

Other aspects and embodiments of the disclosure will be apparent in light of the following detailed description.

### BRIEF DESCRIPTION OF THE DRAWINGS

FIG. 1 is a schematic illustration of an exemplary experimental workflow, including centrifugal-force-driven alginate microgel synthesis, followed by ion-exchange (IX), and the subsequent microgel swelling and cargo release. Inset (middle-bottom panel): optical micrograph of as-prepared alginate microgels post IX. Scale bar: 500 μm. Inset (right panels): fluorescent micrographs of a complex delivery system composed of various number ratios of post-IX microgels containing different dye-labeled dextrans (green and red) for multiplexed cargo release. Scale bars: 1000 μm.

FIG. 2A is optical micrographs of 5Ca5Mg microgels prepared by ion-exchange (IX) and co-ion methods, and IX-5Ca5Na before and after swelling for 30 minutes. Scale bars: 500 μm. FIGS. 2B-2C show normalized volume changes over time of alginate microgels prepared by the IX method (triangles) and the co-ion incubation method (squares) using MgCl₂ (FIG. 2B, triangles) and NaCl (FIG. 2C, triangles). FIG. 2D shows stability ratios of alginate microgels prepared by the IX method using MgCl₂ (left bars) and NaCl (right bars). Inset panels: representative micrographs of the IX-microgels that show broken microgels in optical mode (top) and fluorescent mode (bottom). Scale bars: 500 μm. FIG. 2E shows normalized volume change of the IX-5Ca5Mg microgels during repeated swelling and deswelling cycles. The swelling cycle data are fitted using Equation 2. Each data point is taken 5 minutes apart.

FIGS. 3A-3G show structure and property changes of alginate microgels prepared via the IX method. FIG. 3A shows degrees of crosslinking change as a function of incubation time in IX solutions. FIG. 3B is IX kinetics for the IX-1Ca5Mg and IX-5Ca5Mg samples as measured by the average Ca2+ concentration inside the microgels tracked as a function of incubation time. The SEM micrographs are representative images of freeze-dried IX-1Ca5Mg at different incubation times. Scale bars (0 min): 500 μm. Scale bars (the rest): 1 mm. Inset: EDS mapping of Ca signal. Scale Bars: 500 μm. FIG. 3C shows roughness change ratio, defined as relative change in the standard deviations of microgels' pixel intensity analyzed by ImageJ, of the IX-xCa5Mg microgels plotted as a function of the degree of alginate crosslinking, which can be further correlated with incubation time. Error bar represents standard deviation of the roughness change ratio (n=5). The SEM micrographs are representative images of freeze-dried IX-1Ca5Mg at different incubation times. Scale bars: 200 μm. FIG. 3D shows a comparison of roughness between microgels prepared by different methods. Error bar represents standard deviation of the pixel intensity fluctuation (n=5). FIGS. 3E-3F show MSD values analyzed using trajectories of 30 nm latex beads in IX-1Ca5Mg (FIG. 3E) and IX-1Ca5Na (FIG. 3F) alginate microgels during the IX process and in DI water. FIG. 3G shows the mean MSD values (left ordinate) and calculated diffusivities (right ordinate) change as a function of IX time and exposure time to DI water.

FIGS. 4A-4B show cargo release profiles of Rhodamine B (RhB) and methylene blue (MB) from the IX-1Ca5x (FIG. 4A) and IX-10Ca5x (FIG. 4B) samples (x=Mg or Na). Error bars represent the standard deviation in measured concentration (n=3). Dotted lines represent the fitted release profiles using Equation 5. FIG. 4C shows the cargo release profiles of 3 kDa and 70 kDa dextrans from the IX-xCa5Na microgels (x=5 or 10). Error bars represent the standard deviation in measured fluorescent intensity (n=3).

FIGS. 5A-5H show complex release systems multiplexing different ion-exchanged microgels. FIG. 5A shows deconvoluted release profiles of RhB (top) and MB (bottom) from three microgel systems consisting of different compositions of the IX-1Ca5Mg microgels preloaded with either RhB or MB. Dotted lines represent the best fits from Equation 5. FIG. 5B is representative UV-Vis spectra of the RhB and MB payloads released from the three microgel compositions used in FIG. 5A at 20 minutes of release. Inset: optical micrographs of microgels encapsulated with different dyes mixed in different compositions. Scale bar: 500 μm. FIG. 5C is fitted payload release rate constants (left ordinate) for both RhB and MB, as well as the ratios of the weight of RhB-encapsulated microgels (W_{RhB}) to that of MB-encapsulated microgels (W_{MB}) (right ordinate) for all three microgel compositions tested in FIG. 5A. Error bars represent standard deviations of presented data (n=3). FIG. 5D shows fitted release rates as a function of time and the corresponding release profiles (FIGS. 5E-5G) for the fast-(IX-5Ca5Mg), intermediate-(IX-5Ca5Na), and slow-(IX-10Ca5Na) releasing microgels. Error bars represent standard deviations of presented data (n=3). FIG. 5H shows the fitted release rates of three microgel compositions consist of the fast-releasing regime (light grey, short dashes) and a slow-releasing one (dark grey, short dashes).

### DETAILED DESCRIPTION

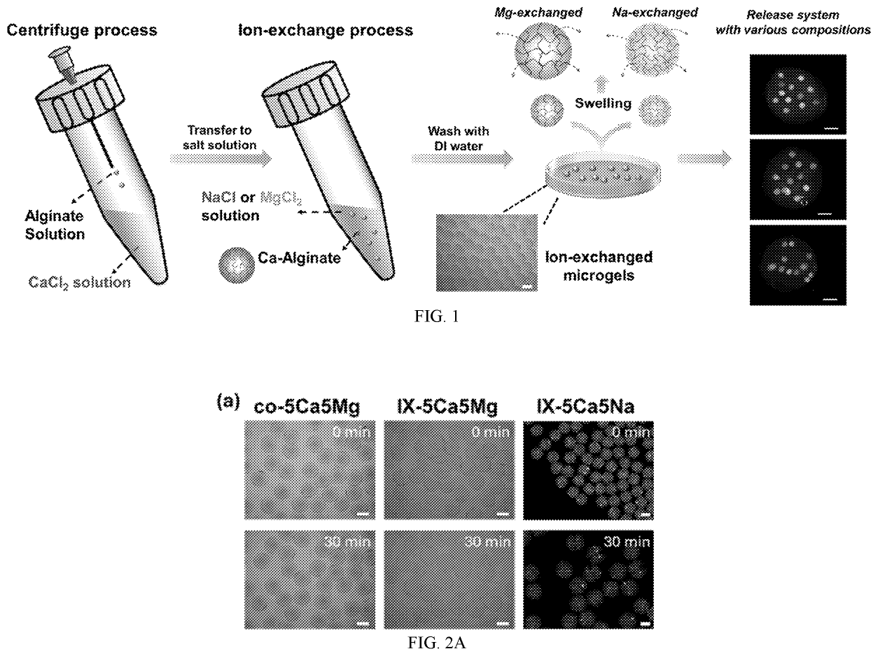

Described herein are scalable methods to prepare alginate hydrogels, e.g., alginate microgels, with programmable release profiles via an ion-exchange (IX) method (FIG. 1). Exemplary microgels are formulated under a first set of crosslinking conditions, for example, using a centrifugal-force-driven technique, and subsequently transferred to a second set of crosslinking conditions, e.g., various salt solutions for IX. By adjusting the type and concentration of cations used during the IX process, the crosslinking density and swollen mesh size of the as-prepared microgels is controlled. The methods are amenable to high throughput at low cost. By choosing the nature of the crosslinkers, both for the initial formation and the IX process, the particle stability and swelling of the hydrogels can be controlled to a desired level based on downstream applications and uses.

Using both diffusion and entrapment to incorporate active agents into the hydrogel matrix, hydrogels, or systems comprising the hydrogels, can be tailored to specific drug release profiles. As such, these methods enable the development of hydrogels with tunable release performance that is compatible with payloads of varying molecular weights and charge characteristics, without resorting to complex crosslinking chemistries and polymer designs. The alginate hydrogels with different release profiles can be further combined in modular delivery systems with programmable dosing control, offering opportunities for biocompatible delivery systems and methods for engineering microcapsule-based therapeutics for diverse biomedical applications.

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular. All publications, patent applications, patents and other references mentioned herein are incorporated by reference in their entirety.

In those instances where a convention analogous to “at least one of A, B, and C, etc.” is used, in general such a construction is intended in the sense one having skill in the art would understand the convention (e.g., “a composition comprising at least one of A, B, and C” would include but not be limited to compositions that comprise that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As used herein, and unless stated otherwise, the terms “by weight”, “w/w”, “weight percent”, or “wt. %”, which are used herein interchangeably, describe the concentration of a particular substance out of the total weight of the corresponding mixture, solution, formulation or composition.

The term “hydrogel” herein refers to a specific type of gel in which water-swellable polymeric matrices that can absorb a substantial amount of water in a three-dimensional network of macromolecules held together by covalent or noncovalent crosslinks.

By “active agent” it is meant an entity, a substance or a chemical capable of producing an effect. For example, any agent or agents suitable for delivery to a subject with the present hydrogel or hydrogel systems to produce an effect may be used. The active agent may comprise a pharmaceutical agent, a macromolecule, a diagnostic substance (e.g., a contrast or imaging agent), a nutritional substance, a cell, or a combination thereof.

The term “release” means the processes by which at least about 50% of the active agent, e.g., or therapeutic agent, are transferred from their initial position in the hydrogel comprising thereof to the surrounding medium.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., humans and non-humans) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

As used herein, the terms “providing,” “administering,” and “introducing,” are used interchangeably herein and refer to the placement into a subject by a method or route which results in at least partial localization to a desired site. Administration can be by any appropriate route which results in delivery to a desired location in the subject.

As used herein, “treat,” “treating,” and the like means a slowing, stopping, or reversing of progression of a disease or disorder. The term also means a reversing of the progression of such a disease or disorder to a point of eliminating or greatly reducing the symptoms. As such, “treating” means an application or administration of an agent to a subject, where the subject has a disease or a symptom of a disease, where the purpose is to cure, heal, alleviate, relieve, alter, remedy, ameliorate, improve, or affect the disease or symptoms of the disease.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. METHODS

In one aspect, disclosed here are methods for modifying an alginate hydrogel. The alginate hydrogel may be modified to confer one or more desired characteristics, e.g., swelling rate, swelling ratio, stability, crosslinking strength and/or density, cargo release profile, or any combination thereof, not embodied or possessed by the original or existing alginate hydrogel.

The methods comprise incubating an alginate hydrogel in a solution comprising a second crosslinker comprising a different cation or cation mixture from the first crosslinker (e.g., first cation or cation mixture) used to form the original or initial alginate hydrogel being modified.

Alginate is a linear anionic polysaccharide derived from brown algae or bacteria. The term “alginate” is used herein to include algin, salts of alginic acid, derivatives of alginic acid, alginic acid itself or any derivative thereof. Alginate consists of repeating units of β-1,4-linked D-mannuronic acid (M) and L-guluronic acid (G) in varying ratios. The M/G ratio depends on seaweed species and the part of the plant from which the alginate has been extracted. Exemplary seaweeds from which alginate may be derived include but are not limited to Laminaria hyperborea, Lessonia trabeculata, Lessonia nigrescens, Laminaria digitata, Macrocystis pyrifera, Ascophyllum nodosum, Laminaria japonica, antarctica, Durvillea potatorum, and Eckonia maxima. Alginate hydrogels are hydrophilic, biocompatible, and non-immunogenic, making them well-suited for biomedical engineering and clinical applications. Typically, these hydrogels are formed by ionic crosslinking using divalent cations such as Ca²⁺.

In some embodiments, the alginate hydrogel is an alginate microgel. Alginate microgels a micrometer-sized spherical particles of crosslinked alginate. Spherical alginate gels are created by contacting aqueous droplets of alginate with divalent cations, e.g., Ca²⁺. Methods suitable for use in generating an alginate microgel include emulsion-based methods, a microfluidic methods, lithography, or a centrifugal-force-driven methods. In select embodiments, alginate hydrogels as described herein are alginate microgels generated by delivering alginate (e.g., from a 5 wt % solution) using a centrifugal-force-driven method into a first crosslinker, as described in the examples below. The size of the particles in the microgel can be controlled by rotation speed and the aperture of the alginate delivery devices (e.g., a syringe or nozzle). Such a generated alginate microgel can then be subject to the disclosed methods in which the alginate microgel is incubated in a solution of a second crosslinker, comprising a different cation or cation mixture from the first crosslinker.

Thus, the methods may further comprise generating an alginate hydrogel, e.g., an alginate microgel, with a first crosslinker. In some embodiments, the methods further comprise adding alginate to a solution comprising the first crosslinker to form an initial alginate hydrogel, e.g., an alginate microgel, prior to incubating the initial alginate hydrogel in a solution comprising the second crosslinker.

The nature of the first crosslinker as compared to the second crosslinker, both in identity and concentration, can be modulated based on the desired characteristics of the resulting hydrogel. The methods are not limited to the exact nature or difference between the first and second crosslinkers. Alginate can be crosslinked by a number of mono-, di-, tri-, and tetravalent cations (e.g., Ag⁺, Na⁺, Mg²⁺, Ca²⁺, Sr²⁺, Ba²⁺, Mn²⁺, Co²⁺, Ni²⁺, Cu²⁺, Zn²⁺, Cd²⁺, Pb²⁺, UO₂²⁺, Cr³⁺, Fe³⁺, Al³⁺, Ga³⁺, Y³⁺, La³⁺, Ce³⁺, Nd³⁺, Eu³⁺, Tb³⁺, Gd³⁺, Zr⁴⁺, Th⁴⁺), any of which may be used in the first or second crosslinker alone or in combination.

For example, a first crosslinker may comprise a single cation and the second crosslinker then comprises another cation crosslinker(s), or alternatively the first crosslinker may comprise a combination of cations, such that second crosslinker may comprise a different mixture of cations, either in identity, concentration, or a combination thereof. In instances in which the first crosslinker comprises calcium, alone or in combination with one or more other cations, the second crosslinker may include magnesium or sodium, e.g., in place or calcium, or merely lack calcium.

In some embodiments, the alginate hydrogel (e.g., alginate microgel) is generated by adding alginate to a solution comprising calcium to form an initial alginate hydrogel. In select embodiments, alginate hydrogel is generated by adding alginate to a solution comprising about 0.1 to about 10 wt % calcium (e.g., about 0.1 wt %, about 0.5 wt %, about 1 wt %, about 2 wt %, about 3 wt %, about 4 wt %, about 5 wt %, about 6 wt %, about 7 wt %, about 8 wt %, about 9 wt %, or about 10 wt %). In exemplary embodiments, the alginate hydrogel is generated by extruding small droplets of alginate (e.g., from a syringe) into a solution comprising about 0.1 to about 10 wt % calcium using a centrifugal-force-driven method.

In some embodiments, an initial alginate hydrogel generated with a first linker comprising calcium is incubated with a solution of second crosslinker lacking calcium. In select embodiments, the second crosslinker is magnesium or sodium. The second crosslinker, e.g., magnesium or sodium, may be provided in a solution, e.g., a solution comprising a 0.1 to 10 wt % magnesium or sodium (e.g., about 0.1 wt %, about 0.5 wt %, about 1 wt %, about 2 wt %, about 3 wt %, about 4 wt %, about 5 wt %, about 6 wt %, about 7 wt %, about 8 wt %, about 9 wt %, or about 10 wt %).

In some embodiments, an initial alginate microgel generated with a first crosslinker comprising calcium is incubated with a solution of second crosslinker lacking calcium. In select embodiments, the second crosslinker is magnesium or sodium. The second crosslinker, e.g., magnesium or sodium, may be provided in a solution, e.g., a solution comprising a 0.1 to 10 wt % (e.g., about 0.1 wt %, about 0.5 wt %, about 1 wt %, about 2 wt %, about 3 wt %, about 4 wt %, about 5 wt %, about 6 wt %, about 7 wt %, about 8 wt %, about 9 wt %, or about 10 wt %) magnesium or sodium.

In some embodiments, the alginate hydrogel comprises an active agent. Thus, in some embodiments, the methods comprise incubating an alginate hydrogel, comprising or having an active agent dispersed within its matrix, in a solution comprising a second crosslinker comprising a different cation or cation mixture from the first crosslinker (e.g., first cation or cation mixture) used to form the original or initial alginate hydrogel being modified.

In such embodiments in which the initial hydrogel is being generated as a part of the disclosed methods, the active agent can be added to the alginate hydrogel during generation, such as for example, adding an active agent to the alginate or first crosslinker solution, in the methods described above. Alternatively or in addition, the alginate hydrogel formed with the first crosslinker may be incubated with a solution comprising the active agent for loading or intercalating the active agent throughout the matrix of the hydrogel, e.g., by passive diffusion. Thus, in some embodiments, the methods may further comprise loading an alginate hydrogel with an active agent prior to incubating in a solution comprising the second crosslinker.

An active agent may be loaded into the alginate hydrogel during or after incubating in the solution comprising the second crosslinker. In some embodiments, the solution comprising the second crosslinker further comprises an active agent. Thus, the active agent intercalates into and is entrapped in the hydrogel during the incubation with the second crosslinker. In some embodiments, the method further comprises incubating the modified alginate hydrogel with an active agent. Thus, the modified alginate hydrogel formed after the incubation with the second crosslinker is subsequently loaded with an active agent.

In some embodiments, the methods further comprise rinsing the alginate hydrogel and/or swelling the alginate hydrogel following incubation with the second crosslinker. Rinsing and swelling may be done prior to use of the hydrogel, prior to loading with an active agent, or immediately following the incubation with the second crosslinker (e.g., to remove excess cations from the solution surrounding the hydrogel).

In some embodiments, the methods further comprise coating at least a portion of the alginate hydrogel, or particles thereof as in an alginate microgel. In some embodiments, the coating is simultaneous to the incubation with the second crosslinker, or following incubation with the second crosslinker, rinsing, and/or swelling. Any coating agent for use with hydrogels, specifically alginate hydrogels, is suitable for use with the disclosed methods. The coating may be selected based on the intended use or a specific property being modulated, such as mechanical strength, degradation rate, bioactivity, or cell interactions. Exemplary coating agents include, but are not limited to, polymers such as polyethylene glycol (PEG), polydimethylsiloxane (PDMS), chitosan, poly(lactic-co-glycolic acid) (PLGA), or biopolymers including collagen and fibrinogen, peptides such as RGD peptides or poly-lysine.

### 3. ALGINATE HYDROGELS AND SYSTEMS

Also disclosed herein are alginate hydrogels (e.g., alginate microgels) comprising modified cation crosslinks to confer a desired swelling rate, swelling ratio, stability, crosslinking strength and/or density, cargo release profile, or combination thereof. In some embodiments, the alginate hydrogels are generated or formed by the methods described above. In some embodiments, the modified hydrogels comprise one or more active agents. In some embodiments, these modified hydrogels or particles thereof, in the context of alginate microgels, are at least partially coated, e.g., with a coating agent as described above.

Further disclosed herein are hydrogel systems comprising at least one alginate hydrogel (e.g., alginate microgel) as described herein. In some embodiments, the hydrogel system comprises two or more alginate hydrogels (e.g., alginate microgels) as described herein. In some embodiments, the two or more alginate hydrogels may contain different swelling rates, swelling ratios, stabilities, crosslinking strengths and/or densities, cargo release profiles, or combination thereof from other alginate hydrogels in the system. For example, the two or more alginate hydrogels may be prepared or generated using different second crosslinkers, as described in the methods above. Alternatively, the two or more alginate hydrogels may be prepared or generated from different starting alginate hydrogels (e.g., hydrogels initially crosslinked with a different first crosslinker).

Alternatively, or in addition, any or all of the at least one alginate hydrogels may be at least partially coated, e.g., with a coating agent as described above. Thus, the at least one alginate hydrogel may comprise a single type of alginate hydrogel having a desired swelling rate, swelling ratio, stability, crosslinking strength and/or density, cargo release profile, or combination thereof, but have two or more different coatings, e.g., uncoated, coated with a first agent, coated with a second agent, to provide different mechanical strength, degradation rate, bioactivity, or cell interactions.

Alternatively, or in addition, any or all of the at least one alginate hydrogels may comprise at least one active agent. For example, different alginate hydrogels may have the same active agent or different types of alginate hydrogels (in terms of their swelling rates, swelling ratios, stabilities, crosslinking strengths and/or densities, cargo release profiles, or combination thereof) may be used for different active agents. In some embodiments, one or more of the at least one alginate hydrogels may be selected based on swelling rate, swelling ratio, stability, crosslinking strength and/or density, cargo release profile, or combination thereof for individual active agents. In some embodiments, one or more of the at least one alginate hydrogels may include the same active agent but with modified swelling rate, swelling ratio, stability, crosslinking strength and/or density, cargo release profile, mechanical strength, degradation rate, bioactivity, and/or cell interactions to create the desired system for delivering the active agent over a desired time or in a desired concentration. As such, in some embodiments, the hydrogel system can enable both rapid and sustained release of the active agent in a single system.

The alginate hydrogels or systems may incorporate a “therapeutically effective amount” or a “prophylactically effective amount” of an active agent, e.g., a therapeutic agent. A “therapeutically effective amount” refers to an amount effective, at dosages and for periods of time necessary, to achieve the desired therapeutic result. The therapeutically effective amount may be determined by a person skilled in the art and may vary according to factors such as the disease state, age, sex, and weight of the individual, and the ability of the composition to elicit a desired response in the individual. A therapeutically effective amount is also one in which any toxic or detrimental effects are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The alginate hydrogels or hydrogel systems may be incorporated into pharmaceutical compositions and formulations which include pharmaceutically acceptable carriers. The term “pharmaceutically acceptable carrier,” as used herein, means a non-toxic, inert solid, semi-solid or liquid filler, diluent, encapsulating material, surfactant, cyclodextrins or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; surfactants such as, but not limited to, cremophor EL, cremophor RH 60, Solutol HS 15 and polysorbate 80; cyclodextrins such as, but not limited to, alpha-CD, beta-CD, gamma-CD, HP-beta-CD, SBE-beta-CD; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### 4. USES

The modified alginate hydrogels, or hydrogel systems comprising thereof, as described above disclosed herein, find use in a variety of applications including implants, tissue regeneration and repair, delivery of active agents (e.g., spatial precision or temporal delivery of a single agent or multiple agents, personalization of drug therapies), and the like.

The disclosure provides methods of delivering an active agent to a subject in need thereof, comprising administering to the subject a modified alginate hydrogel, or a hydrogel system comprising thereof, as described above.

The alginate hydrogels or hydrogel systems may be formulated for any appropriate manner of administration, and thus administered, including for example, oral, nasal, intraocular, intravenous, intravaginal, epicutaneous, sublingual, intracranial, intradermal, intraperitoneal, subcutaneous, intramuscular administration, or via inhalation. Techniques and formulations may generally be found in “Remington's Pharmaceutical Sciences,” (Meade Publishing Co., Easton, Pa.). Therapeutic or pharmaceutical compositions must typically be sterile and stable under the conditions of manufacture and storage. In some embodiments, the alginate hydrogels or hydrogel systems are administered by injection.

### 5. KITS

In another aspect, the disclosure provides kits comprising an alginate hydrogel, crosslinker, e.g., crosslinker solutions, a modified alginate hydrogel, or a hydrogel system for use in the methods for modifying an alginate hydrogel or methods of use. The kits may further comprise active agents and/or coating agents as described above. The kits may further comprise components for using the kit, e.g., syringes, tubes, centrifuges, and the like.

The kits can also comprise instructions for using the components of the kit, e.g., for use in the methods for modifying an alginate hydrogel or methods of use. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods or compositions disclosed herein. The kits optionally may provide additional components such as buffers and disposable single-use equipment (e.g., syringes, tubes).

The kits provided herein are in suitable packaging. Suitable packaging includes, but is not limited to, vials, bottles, jars, flexible packaging, and the like. Individual member components of the kits may be physically packaged together or separately.

### 6. EXAMPLES

### Materials and Methods

Materials Sodium alginate (NaAlg), calcium chloride (CaCl₂), 96%), magnesium chloride (MgCl₂, 98%), sodium chloride (NaCl, 99%), and polyethylene glycol (PEG, Mn=6 kDa, 20 kDa), Rhodamine B (RhB, ≥95%), and methylene blue hydrate (MB) were purchased from Sigma-Aldrich Chemicals. Dextran (neutral, Mn=3 kDa, 70 kDa) was purchased from ThermoFisher. All chemical regents were used as received without any further purification.

Preparation of alginate microgels The alginate microgels were prepared by a modified centrifugal-force-driven method (FIG. 1). The centrifugal-force-driven modular micronozzle devices were designed based on reported work (Kang, S.-M., et al., Sci Rep 9, 12776 (2019)). In brief, a 23-gauge syringe needle was inserted into a 5-mL centrifuge tube filled with 0.6 mL of aqueous solution with designated compositions. The connection between the tube cap and needle was sealed with epoxy resin to prevent shaking during the centrifuge process. Next, 100 μL of 5 wt % NaAlg aqueous solution was loaded in the needle. The centrifugation was operated at 500 rpm for 5 minutes (mins), during which the viscous NaAlg solution was extruded through the micronozzle and formed microdroplets in the solution. For the subsequent ion-exchange (IX) step, after centrifugation, all alginate microgels were first crosslinked in an aqueous solution of CaCl₂) for 5 mins. These crosslinked alginate microgels were washed with de-ionized (DI) water, then transferred to a MgCl₂ or NaCl aqueous solution and incubated for 3 hours. The prepared microparticles were named based on the type of salt used during the IX process (MgCl₂ and NaCl), and the concentration of CaCl₂). For instance, if alginate microgels are first collected in a 1 wt % CaCl₂ solution and then incubated in a 5 wt % MgCl₂ solution, they are labeled as IX-1Ca5Mg. To prepare alginate microgels by the co-ion incubation method, the extruded alginate particles were incubated in co-ion baths containing CaCl₂) and MgCl₂ or NaCl at different weight ratios (1:5 or 5:5) at room temperature. The nomenclature follows the IX samples, but sample names have co- as prefix instead of IX-. For example, the alginate microgels incubated in co-ion bath containing 1 wt % CaCl₂ and 5 wt % NaCl are named co-1Ca5Na. In cases where multiple parallel conditions are consolidated into one sample group, “x” is used to denote the parameter that changes. For instance, “co-xCa5Mg” corresponds to microgels prepared using the co-ion incubation method with various concentrations of CaCl₂) and 5 wt % MgCl₂ in the incubation solutions.

Swelling behavior analysis After the IX step, the microgels were washed twice using DI water before being placed on a glass slide under an optical microscope for the swelling tests. DI water was added to initiate swelling. Images of microgels were captured at different time intervals to monitor the size change.

Single particle tracking for mean square displacement (MSD) measurement Fluorescent latex beads (D=30 nm) were loaded into the microgels during the jetting process for single particle tracking analysis. Time-dependent trajectories of latex beads within the microparticles were recorded using the Echo Revolve fluorescent microscope and analyzed using ImageJ. Mean square displacement (MSD) values were calculated using an open-source MATLAB code, via the following equation:

$$MSD ( τ ) = {〈 [ r ( t + τ ) - {r ( t )}^{2} ] 〉}_{t} \qquad ( 1 )$$

where τ measures the time interval between measurements, r(t) marks the position of the particle at absolute time t, and [symbol] ₜ calculates the temporal average of the argument inside.

Controlled cargo release To characterize the release process of the as-prepared alginate microgels, RhB, MB and dextrans of different molecular weights were used as model drugs, with RhB and MB loaded into the microgels during the IX process and dextrans loaded along with the NaAlg precursor before crosslinking. The microgels were jetted into CaCl₂ solutions as previously described. During IX, the RhB and MB dye molecules were simultaneously infused into the microgels at a non-swelled state via passive diffusion. 0.2 mL of the 5 mg/mL dye stock solutions were added into 10 mL of a 5 wt % NaCl or MgCl₂ solutions to prepare the 0.1 mg/mL RhB or MB loaded IX solutions, while the 0.2 mg/mL dextrans were mixed into the NaAlg precursor solutions to prepare the dextrans-loaded microgels. After the IX process is completed, a fixed weight of microgels were transferred into 2 mL of DI water to characterize the payload release process. For both RhB and MB, the absorbance of the solution was monitored using UV-Vis spectroscopy, whereas for 3 kDa and 70 kDa dextrans, the fluorescent intensity of the microgels was integrated for each microgel particle, upon background noise subtraction.

Chemical and morphological characterizations The morphology of the prepared microgels was analyzed using field emission scanning electron microscopy (FE-SEM). Elemental mapping was measured using energy dispersive X-ray spectroscopy (EDS, Oxford EDS system). All samples were freeze-dried overnight before the subsequent characterization experiments.

### Example 1

### Swelling Behavior of Alginate Hydrogel Microgels

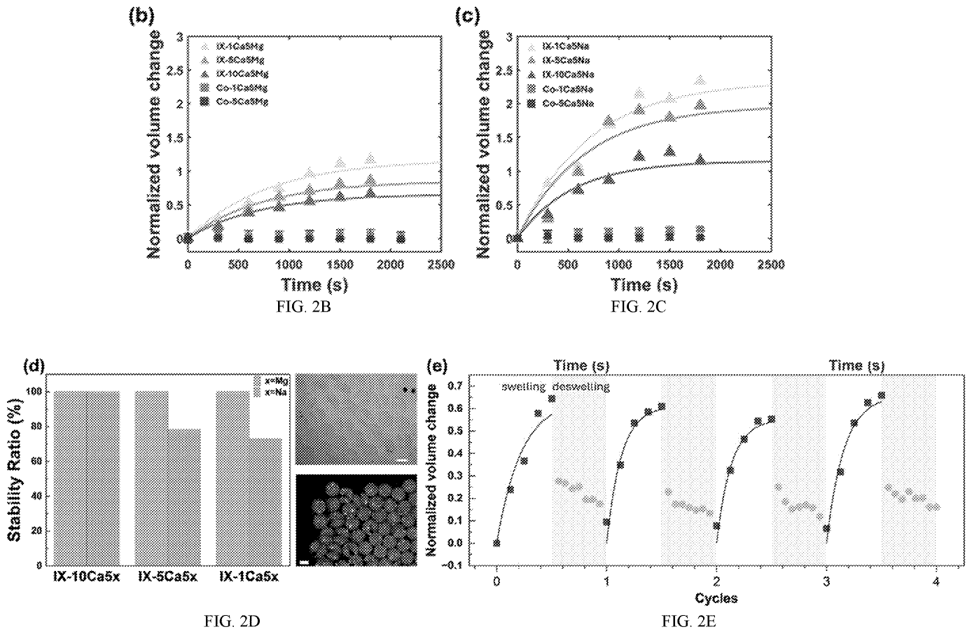

Microgels formulated via a co-ion incubation method exhibited a limited swelling ratio (FIGS. 2B-2C). These microgels were prepared using a centrifugal-force-driven method and were collected in co-ion baths composed of CaCl₂) mixed with either MgCl₂ or NaCl at different concentrations. Mixtures of CaCl₂) with MgCl₂ or NaCl at weight ratios of 1:5 and 5:5 were tested. Upon 3 hours (h) of co-ion incubation, the microgels were transferred to de-ionized (DI) water for swelling tests, and their diameters were measured over time (FIGS. 2B-2C). Only the samples incubated in a CaCl₂-to-NaCl solution at a 1:5 weight ratio (e.g., co-1Ca5Na) exhibited a non-negligible, albeit modest, degree of swelling, while other samples maintained nearly constant size after 40 minutes of DI water exposure (FIG. 2C). After 20 hours of swelling, the microgel volumes increased by approximately 52% (e.g., swelling ratio) for the 1 wt % CaCl₂) co-ion samples and about 33% for those incubated with 5 wt % CaCl₂). Neither the extent nor the rate of swelling was significantly influenced by the co-ion identity (e.g., Mg²⁺ vs. Na⁺). As a control, microgels crosslinked solely with Ca²⁺ do not swell at all when exposed to DI water. The modest swelling observed in co-ion incubated microgels is attributed to the competition between Mg²⁺ or Na⁺ ions with Ca²⁺ for the crosslinking sites within the alginate network. Ca²⁺ ions, due to their stronger affinity with the carboxylate groups, dominate the crosslinking process. As a result, the alginate microgels crosslinked in co-ion baths are chemically similar to those crosslinked with neat CaCl₂ solutions at a similar concentration, limiting the applicability of using co-ion incubation for alginate microstructure tuning.

On the other hand, the results suggest that both the swelling rate and final swollen size of the microgels in DI water could be further tuned using an ion-exchange (IX) method. Microgels subjected to IX exhibited significant swelling within 30 minutes of DI water exposure (FIGS. 2B-2C). All microgels tested gradually swelled until reaching a size plateau, with their final swollen radii varying based on the type and concentration of salt used during the jetting and subsequent IX processes. Among the MgCl₂-exchanged samples, those initially crosslinked in 10 wt % CaCl₂) and then subjected to IX in 5 wt % MgCl₂ (e.g., IX-10Ca5Mg) swelled less than those crosslinked with 5 wt % CaCl₂ (IX-5Ca5Mg), with the 1 wt % CaCl₂) crosslinked samples (IX-1Ca5Mg) swelling the most (FIG. 2B). The higher initial Ca²⁺ concentration in the IX-10Ca5Mg sample likely limits the extent of Mg²⁺ exchange, thereby preserving Ca²⁺'s crosslinking effect, whereas the IX-1Ca5Mg and IX-5Ca5Mg samples start with fewer Ca²⁺ ions. Similarly, the swelling behavior of NaCl-exchanged samples exhibited similar relation with the initial Ca²⁺ crosslinker concentration (FIG. 2C). The swelling kinetics can be modeled as a diffusion-dominated process:

$$\frac{Q}{{Q}_{e}} = 1 - {e}^{- kt} \qquad ( 2 )$$

assuming that the microgels swell isotropically such that the percent increase in radius translates directly to volumetric expansion. Here, Q measures the percent change in volume at time t, Qₑ represents the equilibrium percent change in volume, and k is the swelling rate constant. Comparing the fitted rate constants (Table 1) revealed that the swelling rates differ between the MgCl₂- and NaCl-exchanged samples, likely due to the valency and interaction characteristics of the exchanged cations. Specifically, monovalent Na⁺ interacts with only one free carboxylate group, thereby disrupting the strong Ca²⁺ ionic crosslinks, whereas Mg²⁺, although capable of chelating with two carboxylates like Ca²⁺, preserves the crosslinking effect only partially due to its smaller crystallographic ionic radius. Within each IX group, the initial Ca²⁺ crosslinking density further modulates the swelling rate: samples with lower initial Ca²⁺ content (e.g., IX-1Ca5x) swell the fastest, followed by IX-5Ca5x and then IX-10Ca5x. During the IX process, Mg²⁺ and Na⁺ replace Ca²⁺ as crosslinkers in the alginate backbone. The absence of Ca²⁺ in the IX solution sets up an entropic driving force for the chelated Ca²⁺ to dissociate and diffuse out, thereby weakening the crosslinking structure and resulting in larger swelling ratios compared to the co-ion incubation method (FIGS. 2B-2C).

Due to the increased swelling ratio, the boundaries of NaCl-exchanged alginate microgels in DI water become blurred as time progresses. To better visualize their swelling behavior, 30 nm fluorescent PS beads were incorporated into the alginate microparticles, creating clearly defined green fluorescent areas. Some NaCl-exchanged microgels burst after 30 minutes of DI water exposure, whereas all MgCl₂-exchanged microgels remained intact, demonstrating higher stability (FIG. 2D). The stability ratio was defined as the number of intact microgels after 30 minutes of swelling in DI water divided by the total number of microgel particles at the start of the swelling experiment. Microgels crosslinked with a higher initial CaCl₂) concentration exhibited a higher stability ratio than those crosslinked with lower CaCl₂) concentrations (FIG. 2D). For NaCl-exchanged microgels, for instance, the stability ratio decreased from 100% to 73% as the CaCl₂) concentration decreased from 10 wt % to 1 wt %. This indicated that the initial CaCl₂) crosslinker concentration before IX can be used to fine-tune microgel stability.

The reversibility of swelling behavior in IX-5Ca5Mg microgels was further examined by subjecting them to repeated cycles of swelling in DI water (20 minutes) followed by deswelling in 5 wt % CaCl₂) (30 minutes). The microgels underwent four complete cycles without significant deterioration in swellability (FIG. 2E). After each deswelling cycle, the microgels were transferred to a 5 wt % MgCl₂ solution for 1 hour to restore their ion-exchanged equilibrium state before returning to DI water for re-swelling. The percent change in volume was modeled using Equation 2, which revealed that the swelling rate for the first cycle was significantly slower than in subsequent cycles. This initial delay may have resulted from the jetting process creating highly entangled alginate chains; repeated swelling likely relaxes this entanglement, leading to faster swelling after the first cycle. This reversible swelling-deswelling behavior suggests that ion-exchanged alginate microgels have potential as drug delivery vehicles capable of repeated therapeutic loading and unloading.

TABLE 1
Fitted swelling rate constants for different IX-microgels (using Equation 2).

| | IX-1Ca5Mg | IX-5Ca5Mg | IX-10Ca5Mg | IX-1Ca5Na | IX-5Ca5Na | IX-10Ca5Na |
|---|---|---|---|---|---|---|
| k(s⁻¹) | 0.00127 | 0.00136 | 0.00142 | 0.00143 | 0.00152 | 0.00184 |
| R² | 0.9596 | 0.9654 | 0.9816 | 0.9905 | 0.9708 | 0.9654 |

### Example 2

### Property Changes of Alginate Microgels During the IX Process

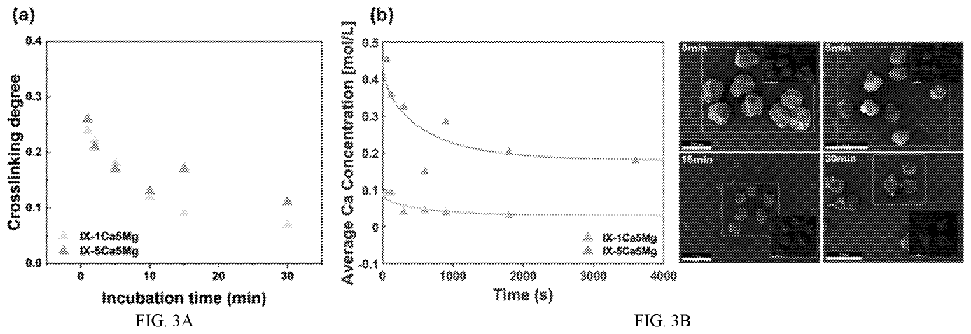

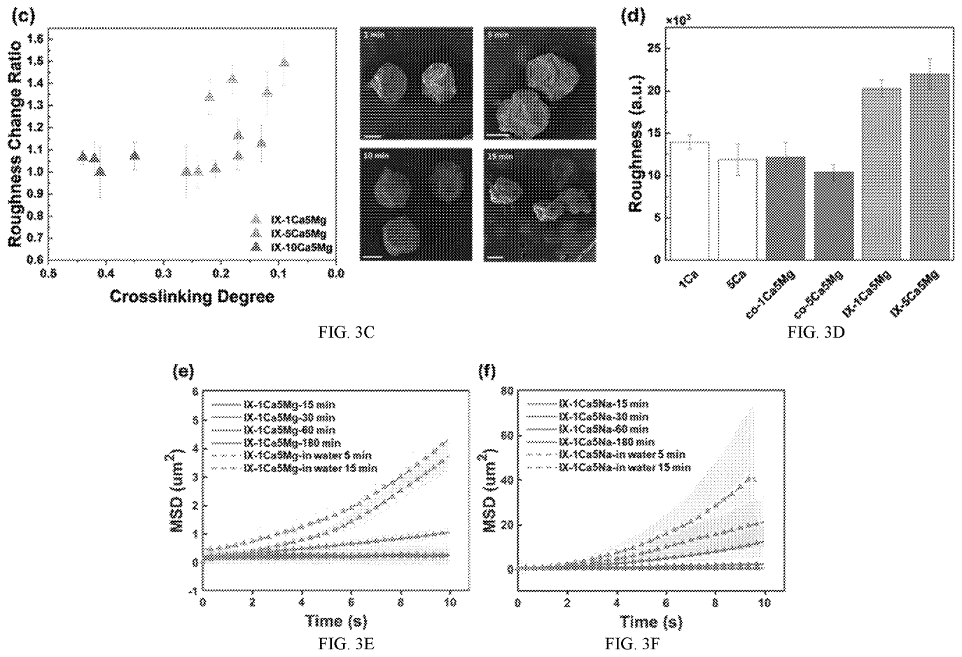

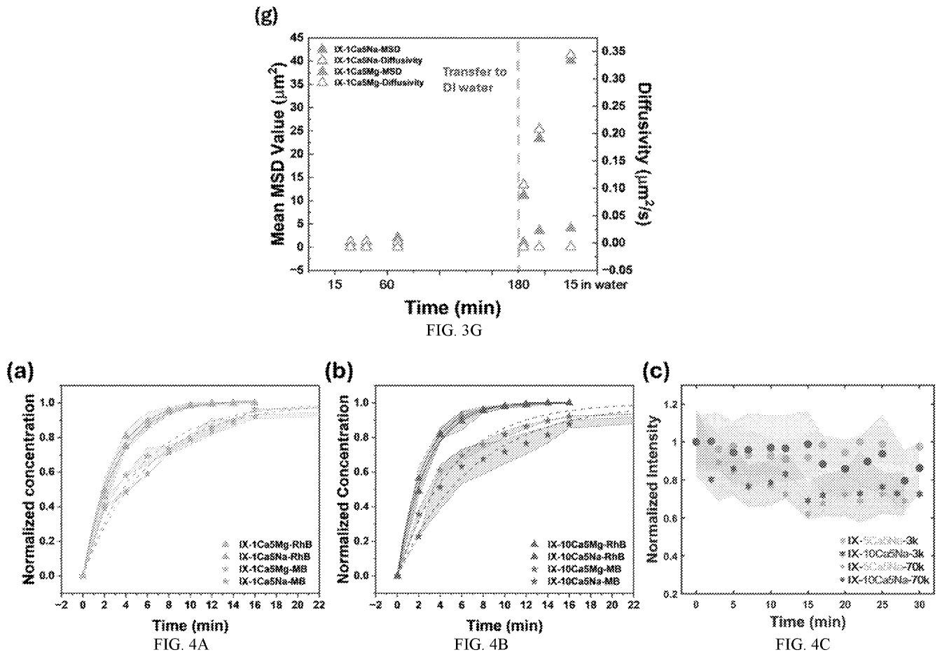

To gain further insight into the IX process, the structure-property relations induced by IX with Na⁺ and Mg²⁺ were characterized. Morphological and elemental analyses were performed on MgCl₂-exchanged samples using scanning electron microscopy (SEM) and energy dispersive X-ray spectroscopy (EDS) mapping to elucidate how the crosslinking density and microgel morphology evolve during IX. Specifically, changes in the Ca²⁺, Mg²⁺ and oxygen contents of the IX-xCa5Mg microgels (where x=1, 5) were analyzed at various incubation times. The alginate content was estimated from the oxygen level using the stoichiometric relation: alginate %=O %/6. As expected, no Mg²⁺ signal was detected in microgels incubated solely in CaCl₂), and the Ca²⁺ content decreased as IX progressed. The degree of crosslinking, calculated from the Ca²⁺ to alginate ratio, decreased with increasing IX time, reaching a plateau after 10 minutes (FIG. 3A). Additionally, particles prepared via the co-ion incubation method exhibited significantly higher degrees of crosslinking. For a quantitative assessment, the IX process was modeled as Fickian diffusion, considering that the ionic radii are relatively small compared to the average pore size of the alginate hydrogel network. It was assumed that the IX reaction rate is fast enough that the overall process is transport-limited. In Equation 3, RCa represents chelated Ca²⁺, while free Ca²⁺ ions are denoted as Ca²⁺. In Equation 4, [Ca²⁺] represents the free Ca²⁺ concentration at space-time (r, t), where r measures the radial distance from the microgel center, and t is the elapsed time. [Ca²⁺]_{∞}, the concentration of the residually bound Ca²⁺ inside the microgel after all the “free” Ca²⁺ ions have diffused out, was experimentally determined and added to the fitted [Ca²⁺] profile to account for the chemical equilibrium between the chelated Ca²⁺ and Mg²⁺.

$$RCa + {Mg}^{2 +} ↔ RMg + {Ca}^{2 +} \qquad ( 3 )$$

$$\frac{\partial [ {Ca}^{2 +} ]}{\partial t} = D \frac{1}{{r}^{2}} \frac{\partial }{\partial r} ( {r}^{2} \frac{\partial [ {Ca}^{2 +} ]}{\partial r} ) \qquad ( 4 )$$

This simple model provided an excellent fit to the experimental data (FIG. 3B). The fitted diffusion coefficients for the interior (Dᵢ) and exterior (Dₑ) of the microgels are as follows: for IX-1Ca5Mg, Dᵢ=1.76×10⁻¹¹ m²/s; for IX-5Ca5Mg, Dₑ=1.14×10⁻¹⁰ m²/s; Dᵢ=1.69×10⁻¹¹ m²/s; Dₑ=1.11×10⁻¹⁰ m²/s. These diffusivities are consistent with reported literature values and indicated no significant differences between the IX-1Ca5Mg and IX-5Ca5Mg samples.

The degree of hydrogel crosslinking is known to affect its structural rigidity. As IX progressed, the microgels' elastic modulus decreased, resulting in elevated surface roughness (due to increased gel buckling) upon freeze-drying. This was evident from the observed intensity fluctuations in the corresponding scanning electron micrographs. With increasing IX time in MgCl₂ (which corresponds to less Ca²⁺-alginate crosslinks) or with lower initial CaCl₂) concentrations, the freeze-dried microgels buckled more easily, leading to increased surface roughness (FIG. 3C). In contrast, microgels prepared via the co-ion incubation method or crosslinked in pure CaCl₂) solutions (without IX) exhibited much smoother surface morphologies (FIG. 3D). Taken together, these results suggest that the ion-exchanged alginate microgels possess fewer Ca²⁺ alginate crosslinks than those prepared by co-ion incubation or non-IX controls, resulting in weaker internal microgel networks, faster swelling rates, and greater swellability in DI water.

Modulating the crosslinking strength and density via the IX process also affected the diffusion properties within the alginate microgels. These properties were evaluated by measuring the Mean Square Displacement (MSD) of encapsulated tracer nanoparticles (FIGS. 3E-3G). Specifically, single particle tracking was used to monitor the MSD of 30 nm fluorescent latex beads in alginate microgels at various stages of IX. Twenty hydrogel microgels containing latex beads (crosslinked in 1 wt % CaCl₂)) were transferred into a microfluidic device, and MgCl₂ or NaCl solution was introduced to initiate the respective IX processes in situ. The movement of the encapsulated latex beads was recorded using a fluorescent microscope, and the MSD values of microgels incubated in IX solution for different times were plotted as a function of time interval between measurements (FIGS. 3E and 3F). For controls, the MSD of latex beads in non-crosslinked alginate aqueous solution and in a neat Ca²⁺-crosslinked alginate gel without IX were measured. The fluorescent beads exhibited rapid movement in the non-crosslinked alginate, whereas samples crosslinked with 1 wt % CaCl₂ showed a drastic reduction in MSD, only about one-tenth of that observed in the non-crosslinked system. Furthermore, exposure of Ca²⁺-crosslinked alginate samples to DI water for 3 hours did not significantly alter the mean MSD values, consistent with the previous observation that the pure Ca²⁺-crosslinked alginate microgels do not swell.

On the other hand, the MSD values of the IX-1Ca5Mg and IX-1Ca5Na samples increased monotonically with IX time, reflecting a decrease in crosslinking density during the IX process (FIG. 3G). Notably, NaCl-exchanged microgels showed a more pronounced increase in mean MSD values compared to their MgCl₂-exchanged counterparts, indicating that they possess weaker crosslinks, consistent with EDS mapping results. After IX was completed, the ionic solutions were removed and replaced with DI water, which induced further swelling and changes in the microgel diffusion properties. Both MgCl₂- and NaCl-exchanged microgels experienced a dramatic increase in mean MSD values due to rapid swelling, with a greater increase observed in the NaCl-exchanged samples (FIG. 3G). The linear portion of the MSD data was used to calculate the tracer particle diffusivities in a three-dimensional system (FIG. 3G), although diffusivities may vary for other small molecular cargos depending on their size and charge. Therefore, the IX process provided a new set of engineering handles to fine-tune cargo diffusivity within ion crosslinked alginate microgels in a facile and biocompatible manner, without resorting to complex chemical reactions.

### Example 3

### Cargo Release Performance of the IX Alginate Microgels

To evaluate the potential of using IX to fine-tune the drug delivery performance of alginate microgels, the release properties of various model compounds encapsulated in the microgels was investigated. The effect of cargo size was assessed by encapsulating molecules of increasing size, Rhodamine B (RhB), 3 kDa dextran, and 70 kDa dextran, either during jet printing or during the IX process. The microgels were then exposed to DI water to initiate the diffusive release of the payloads. Calibration curves for RhB and methylene blue (MB) were obtained via UV-Vis absorption, and the normalized concentration (for RhB) or fluorescent intensity (for dextran) was monitored over time (FIG. 4). The payload release follows first-order kinetics, described by the equation:

$$C ( t ) = {C}_{∞} + A {e}^{- k C} \qquad ( 5 )$$

For small molecular cargo such as RhB, there was no significant difference in the measured release rates between the IX-1Ca5x and IX-10Ca5x microgels (where x=Mg or Na; Table 2). However, an interesting, albeit subtle, trend emerged: samples that swelled faster exhibited slower release rates. This trend was observed when comparing the NaCl- and MgCl₂-exchanged microgels (e.g., IX-1Ca5x and IX-10Ca5x), the 1Ca and 10Ca controls (no IX), and samples ion-exchanged for 24 hours versus 3 hours. This behavior was attributed to the density of free carboxylate groups within the alginate network. At the neutral pH used in the release studies, RhB is slightly cationic; thus, a higher free carboxylate content led to stronger electrostatic interactions with the negatively charged alginate backbone, slowing the release. At the same time, a higher free carboxylate content indicated reduced crosslinking, allowing for increased swelling. However, because RhB is small relative to the network's mesh size, the matrix expansion from swelling did not significantly enhance its diffusion, resulting in this counterintuitive trend. This cargo-alginate electrostatic interaction was further amplified when using methylene blue (MB), which has additional positively charged functional groups (Table 3). Consequently, MB exhibited a slower overall release rate, with NaCl-exchanged microgels releasing MB more slowly than MgCl₂-exchanged ones (FIGS. 4A and 4B).

The release of larger molecular payloads, 3 kDa and 70 kDa dextrans, was studied using the IX-xCa5Na samples (where x=5 or 10) (FIG. 4C). The normalized intensity started at 1 rather than 0 because the data tracked the fluorescent intensity of the microgels, not the surrounding solution. The release rate of 3 kDa dextran was significantly slower than that of RhB and MB, and no significant difference was observed between microgels with different initial Ca²⁺ concentrations. When the molecular weight was increased to 70 kDa, virtually no cargo was released over a similar time scale (FIG. 4C).

TABLE 2
Fitted release rates k, terminal concentration C_{∞}, and fitting parameter A for RhB release of ion-exchanged microgels.

| | IX | | | | | | | |
|---|---|---|---|---|---|---|---|---|
| | 1Ca5Mg | 1Ca5Na | 10Ca5Mg | 10Ca5Na | 1Ca5Mg-24 H | 1Ca5Na-24 H | 1Ca | 10Ca |
| k (min⁻¹) | 0.37 ± 0.018 | 0.32 ± 0.010 | 0.36 ± 0.0056 | 0.37 ± 0.022 | 0.47 ± 0.016 | 0.33 ± 0.0063 | 0.36 ± 0.035 | 0.46 ± 0.024 |
| C_{∞} | 0.00246 | 0.00200 | 0.00279 | 0.00209 | 0.0099 | 0.0098 | 0.00272 | 0.00277 |
| (mg/mL) | | | | | | | | |
| A | −0.0018 | −0.0012 | −0.0020 | −0.0013 | −0.00056 | −0.00050 | −0.0017 | −0.0019 |

TABLE 3
Fitted release rates k, terminal concentration C_{∞}, and fitting
parameter A for MB release of ion-exchanged microgels.

| | IX | | | |
|---|---|---|---|---|
| | 1Ca5Mg | 1Ca5Na | 10Ca5Mg | 10Ca5Na |
| k (min⁻¹) | 0.21 ± 0.029 | 0.14 ± 0.022 | 0.22 ± 0.022 | 0.18 ± 0.040 |
| C_{∞} | 0.000687 | 0.000498 | 0.000703 | 0.00455 |
| (mg/mL) | | | | |
| A | −0.00055 | −0.00030 | −0.00056 | −0.00027 |

### Example 4

### Complex Release System Based on Multiple Alginate Microgels

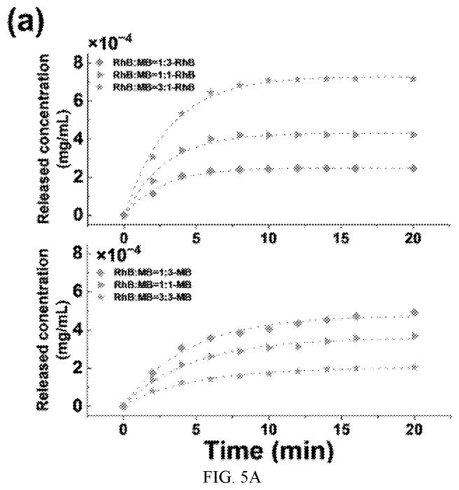

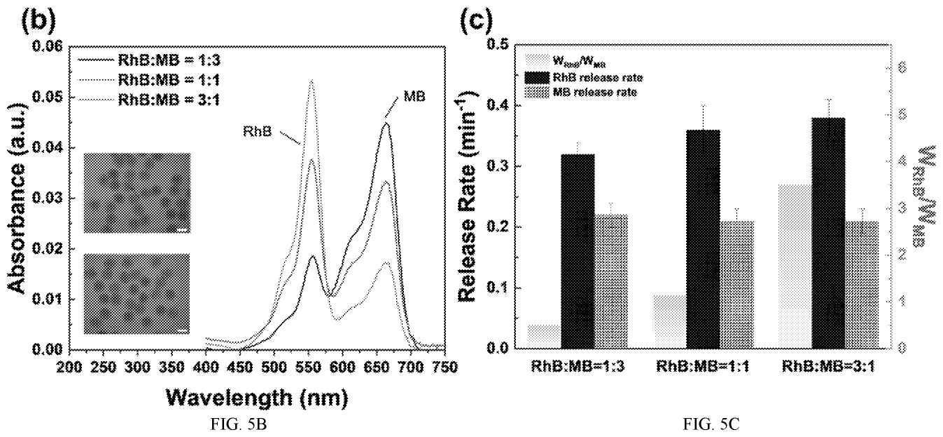

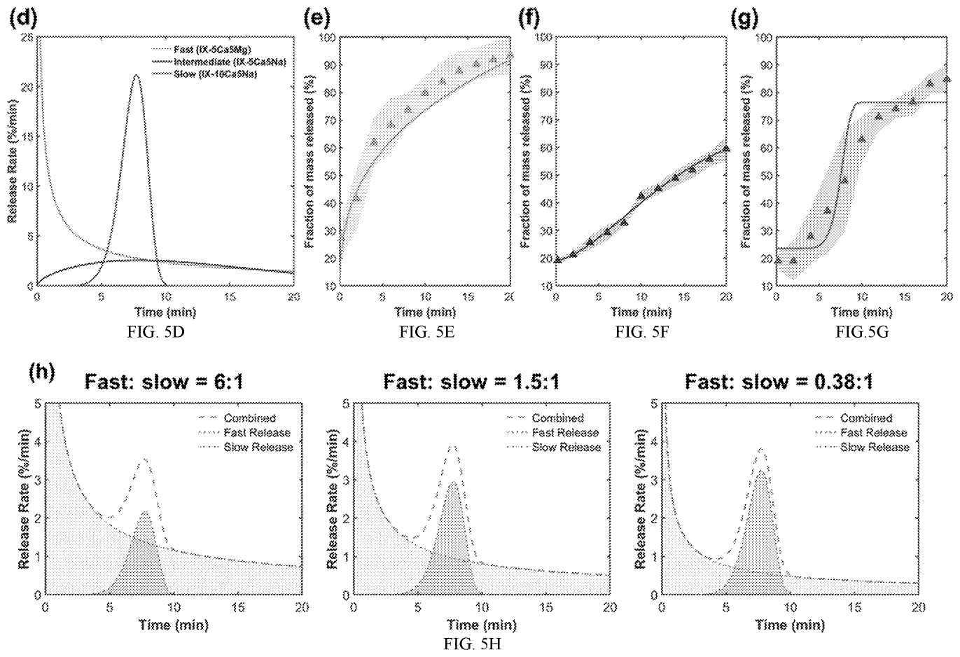

The IX alginate microgels can be combined into ensemble mixtures that encapsulate different molecules, enabling diverse release profiles within a single drug delivery system. To demonstrate this versatility, RhB and MB were used as model drugs with IX-1Ca5Mg microgels as the carrier. All microgel subpopulations were prepared using the method described below. Microgels encapsulating RhB and MB were prepared separately and then mixed at different weight ratios while maintaining a constant total weight of 20 mg. The deconvoluted release profiles revealed a slightly lower encapsulation efficiency for MB compared to RhB, as indicated by the terminal dye concentrations, which aligned with the weight ratios of the two microgel constituents (FIG. 5A). Increasing the proportion of MB-containing microgels led to a higher absorption peak intensity of MB in the supernatant and a corresponding decrease in the RhB peak, as expected (FIG. 5B). Importantly, the release rates of RhB and MB remained consistent with those measured the individual release experiments (FIG. 5C), suggesting that the release of multiple payloads in a single system can be controlled by adjusting the ratios of microgels encapsulating them.

Multiplexed IX alginate microgels can be used to program complex temporal release behavior for a single cargo. To illustrate this, RhB was encapsulated in various IX-microgels as the model drug. The alginate microgels were prepared using different cations during the IX step, and subsequently mixed at varying ratios to form a complex release system. In these experiments, the as-prepared microgels were coated with a polyethylene glycol (PEG) layer to further retard drug release and to help distinguish the differences in RhB diffusion rates among the various IX-microgel populations. To optimize the coating composition, PEG with different molecular weights was added to the IX solution at a total concentration of 5 wt %, while all other procedures remained unchanged. SEM images revealed that the PEG-coated microgels exhibited a smoother surface than the uncoated ones. Next, the release profiles of various PEG-coated IX-10Ca5Na microgels were measured. The microgels coated with a mixture of PEG at a 1:3 ratio (6 kDa to 20 kDa) exhibited a significantly delayed release profile and was used for all subsequent experiments. To quantitatively describe the release process, the overall temporal concentration profile was deconvoluted into two regimes by combining the modified Korsmeyer-Peppas model for burst or sustained release (Equation 6) with the Weibull model for delayed release (Equation 7).

$$\frac{M ( t )}{{M}_{∞}} = k * {t}^{n} + {C}_{1} \qquad ( 6 )$$

where M(t) is the mass of model drug released at time t, M_{∞} is the final equilibrium released mass of drug, k is the rate constant, n is the release exponent, and C₁ is a fitting constant.

$$\frac{M ( t )}{{M}_{∞}} = D * ( 1 - exp ( - α * {t}^{β} ) ) + {C}_{2} \qquad ( 7 )$$

where α is the apparent release constant, β is the shape factor, and C₂ is a fitting constant.

The first-order time derivatives of the release profiles for IX-5Ca5Mg, IX-5Ca5Na, IX-10Ca5Na microgels, reflecting their respective cargo release rates, exhibited peaks at different times, highlighting the potential for a multiplexed system with tunable release times for a single drug payload (FIGS. 5D-5G). The RhB release profile was fitted for the fast-releasing IX-5Ca5Mg microgels using the Korsmeyer-Peppas model and obtained an n value of 0.339, which was lower than expected for non-Fickian diffusion processes (FIG. 5E). This suggested that RhB release in IX-5Ca5Mg microgels was not hindered by interactions with the gel matrix. To demonstrate the feasibility of a complex drug delivery system with tunable release times for a single payload, a pair of the fast-releasing (IX-5Ca5Mg) and slow-releasing (IX-10Ca5Na) microgels were combined. The total number of microgels in each delivery system was kept constant while varying the ratios of these two subpopulations to program the desired release profile (FIG. 5H). The overall release profile exhibits two distinct stages and can be modeled as a linear combination of the Korsmeyer-Peppas and the Weibull equations for individual components:

$$\frac{M ( t )}{{M}_{∞}} = S * D * ( 1 - exp ( - α * {t}^{β} ) ) + F * k * {t}^{n} + {C}_{3} \qquad ( 8 )$$

where S and F are the coefficients corresponding to the proportions of slow- and fast-releasing microgels (Table 4). C₃ is a fitting constant, and the parameters α, β, n, D and k are obtained from Equation 5 and 6.

Reducing the ratio of fast- to slow-releasing microgels led to a corresponding decrease in the fitted S-to-F ratio (Table 4). To visualize the separate contributions of the two subpopulations, the overall payload release rate (calculated from the first-order time derivative of the combined release profile) was decoupled into fast-releasing (orange) and slow-releasing regimes (blue) (FIG. 5H). As the ratio of fast-to-slow microgels decreased, the area underneath the fast-releasing curve diminished, while the slow-releasing contribution increased. Together, these results demonstrated that programmable cargo release can be achieved by tuning the proportions of alginate microgel subpopulations with customizable release profiles via controlled IX.

TABLE 4
Fitted release rates k, terminal concentration C_{∞}, and fitting
parameter A for MB release of ion-exchanged microgels.

| Experimental | | | | | Calculated ratio of |
|---|---|---|---|---|---|
| ratio of fast | | | | | fast and slow- |
| and slow- | | | | | release samples |
| release samples | S*D | F*k | S | F | (ratio of F to S) |
| 6:1 | 5.402 | 15.649 | 0.102 | 0.494 | 4.83:1 |
| 1.5:1 | 7.361 | 10.894 | 0.139 | 0.344 | 2.46:1 |
| 0.38:1 | 8.072 | 6.488 | 0.153 | 0.205 | 1.34:1 |

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

1. A method of modifying an alginate hydrogel comprising:
incubating the alginate hydrogel in a solution comprising a second crosslinker to form a modified alginate hydrogel,
wherein the second crosslinker comprises a different cation or cation mixture from a first crosslinker used to form the alginate hydrogel.

2. (canceled)

3. The method of claim 1, wherein the first crosslinker comprises calcium.

4. The method of claim 3, wherein the first crosslinker further comprises at least one additional cation.

5. The method of claim 1, wherein the second crosslinker comprises magnesium or sodium.

6. The method of claim 3, wherein the second crosslinker lacks calcium.

7. The method of claim 1, wherein the alginate hydrogel is an alginate microgel.

8. The method of claim 1, wherein the method further comprises generating the alginate hydrogel with the first crosslinker.

9. The method of claim 8, wherein generating the alginate hydrogel comprises adding alginate to a solution comprising the first crosslinker.

10. The method of claim 9, wherein the first crosslinker is calcium.

11. The method of claim 10, wherein the calcium is provided in a solution at 1 to 10 wt % calcium.

12. (canceled)

13. The method of claim 1, wherein the alginate hydrogel further comprises an active agent and generating the alginate hydrogel further comprises:
adding the active agent to the first crosslinker or alginate prior to crosslinking;
including the active agent in the solution comprising the second crosslinker; and/or
incubating the modified alginate hydrogel with an active agent.

14-16. (canceled)

17. The method of claim 1, further comprising coating at least a portion of the modified alginate hydrogel or particles thereof.

18. The method of claim 17, wherein the solution comprising the second crosslinker further comprises a polyethylene glycol (PEG).

19. The method of claim 1, wherein the modified alginate hydrogel exhibits modified swelling rates, swelling ratios, stability, crosslinking strength and/or density, cargo release profiles, or combination thereof, as compared to an alginate hydrogel not incubated with the second crosslinker.

20. A modified alginate hydrogel formed by the method of claim 1.

21. The modified alginate hydrogel of claim 20, wherein the modified alginate hydrogel is an alginate microgel.

22. The modified alginate hydrogel of claim 20, wherein the modified alginate hydrogel or particles thereof is at least partially coated with polyethylene glycol (PEG) and/or wherein the modified alginate hydrogel comprises an active agent.

23-24. (canceled)

25. A hydrogel system comprising at least one modified alginate hydrogel or alginate hydrogel of claim 20.

26. (canceled)

27. The hydrogel system of claim 25, wherein:
any one or more or all of the at least one modified alginate hydrogels were prepared with different second crosslinkers;
any one or more or all of the at least one modified alginate hydrogels, or particles thereof, are at least partially coated with polyethylene glycol (PEG); and/or
any one or more or all of the at least one modified alginate hydrogel and/or alginate hydrogel comprises an active agent, wherein each of the at least one modified alginate hydrogel or alginate hydrogel comprises a same or different active agent.

28-30. (canceled)

31. A method of delivering an active agent to a subject in need thereof, comprising administering to the subject a modified alginate hydrogel of claim 20, or a hydrogel system comprising thereof.
